(19) United States
(12) Patent Application Publication
(10) Pub. No.: US 2024/0091241 A1
(43) Pub. Date: Mar. 21, 2024

(54) USE OF CANNABIDIOL PREPARATIONS IN THE TREATMENT OF TEMPORAL LOBE EPILEPSY

(71) Applicant: GW Research Limited, Cambridge (GB)

(72) Inventors: Geoffrey GUY, Cambridge (GB); Volker KNAPPERTZ, Cambridge (GB); Benjamin WHALLEY, Cambridge (GB); Marie WOOLLEY-ROBERTS, Cambridge (GB)

(21) Appl. No.: 17/768,048

(22) PCT Filed: Oct. 23, 2020
(86) PCT No.: PCT/GB2020/052697

§ 371 Date: Apr. 11, 2022

(30) Foreign Application Priority Data
Oct. 25, 2019 (GB) 1915519.1

(51) Int. Cl. A61K 31/00; A61K 36/185; A61P 25/08

(52) U.S. Cl. CPC A61K 31/658; A61K 36/185; A61P 25/08

(57) ABSTRACT

The present invention relates to the use of a cannabidiol (CBD) preparation in the treatment of temporal lobe epilepsy. In particular the CBD preparation is characterized by chemical components and/or functional properties that distinguish them from prior CBD compositions. Preferably the CBD used is in the form of a botanically derived purified CBD which comprises greater than or equal to 98% (w/w) CBD and less than or equal to 2% (w/w) of other cannabinoids. The other cannabinoids present are THC at a concentration of less than or equal to 0.1% (w/w); CBD-Cl at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

### FIELD OF THE INVENTION

The present invention relates to the use of a cannabidiol (CBD) preparation in the treatment of temporal lobe epilepsy. In particular the CBD preparation is characterized by chemical components and/or functional properties that distinguish them from prior CBD compositions.

Preferably the CBD used is in the form of a botanically derived purified CBD which comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w). The botanically derived purified CBD preferably also comprises a mixture of both trans-THC and cis-THC. Alternatively, a synthetically produced CBD is used.

### BACKGROUND TO THE INVENTION

Temporal lobe epilepsy (TLE) is the most common form of focal epilepsy. About 6 out of 10 people with focal epilepsy have temporal lobe epilepsy. Seizures in TLE start or involve in one or both temporal lobes in the brain.

There are two types of TLE:

Mesial temporal lobe epilepsy (MTLE) involves the medial or internal structures of the temporal lobe. Seizures often begin in a structure of the brain called the hippocampus or surrounding area. MTLE accounts for almost 80% of all temporal lobe seizures.

Neocortical or lateral temporal lobe epilepsy involves the outer part of the temporal lobe.

Mesial temporal lobe epilepsy usually begins around age 10 or 20, but it can start at any age. Usually a person has had a seizure with fever or an injury to the brain which is the precursor to a seizure.

Previous names for seizures that occur in TLE, include psychomotor seizures, limbic seizures, temporal lobe seizures, complex partial and simple partial. The commonly used term for these seizures now is focal onset seizures. Focal seizures are then described by whether a person stays awake and aware or has impaired awareness during a seizure.

TLE is treated with antiepileptic medication, however over a third of patients do not gain full seizure control. Additional treatment can include surgery to remove areas of scarring on the brain or the implantation of devices such as the vagus nerve stimulation may be additional treatment options.

The present invention demonstrates an increased efficacy of a botanically derived purified CBD preparation which comprises minor amounts of the cannabinoids CBD-C1, CBDV, CBD-C4 and THC over a synthetic CBD which does not comprise minor amounts of cannabinoids in a rat model of temporal lobe epilepsy. These data are particularly surprising particularly given the fact that the concentration of CBD within the botanically derived purified CBD preparation and the synthetic preparation were the same.

### BRIEF SUMMARY OF THE DISCLOSURE

In accordance with a first aspect of the present invention there is provided a cannabidiol (CBD) preparation for use in the treatment of temporal lobe epilepsy (TLE).

Preferably the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC); cannabidiol-C1 (CBD-C1); cannabidivarin (CBDV); and cannabidiol-C4 (CBD-C4), and wherein the THC is present as a mixture of trans-THC and cis-THC.

Preferably the CBD is present is isolated from cannabis plant material.

More preferably at least a portion of at least one of the cannabinoids present in the CBD preparation is isolated from cannabis plant material.

Alternatively, the CBD is present as a synthetic preparation. In a further embodiment at least a portion of at least one of the cannabinoids present in the CBD preparation is prepared synthetically.

Preferably the dose of CBD is greater than 5 mg/kg/day. More preferably the dose of CBD is 20 mg/kg/day. More preferably the dose of CBD is 25 mg/kg/day. More preferably the dose of CBD is 50 mg/kg/day.

In accordance with a second aspect of the present invention there is provided a method of treating temporal lobe epilepsy (TLE) comprising administering a cannabidiol (CBD) preparation to the subject in need thereof.

### BRIEF DESCRIPTION OF THE DRAWINGS

FIG. 1 shows the drug product manufacturing process.

FIG. 2 shows the assessment of Synthetic (CBD SYN) vs Botanical CBD (CBD BOT) on the PSBB score.

FIG. 3 shows the assessment of Synthetic (CBD SYN) vs Botanical CBD (CBD BOT) in the elevated plus maze test.

FIG. 4 shows the assessment of Synthetic (CBD SYN) vs Botanical CBD (CBD BOT) in the social investigation test.

### DEFINITIONS

Definitions of some of the terms used to describe the invention are detailed below:

Over 100 different cannabinoids have been identified, see for example, Handbook of Cannabis, Roger Pertwee, Chapter 1, pages 3 to 15. These cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

The human equivalent dose (HED) can be estimated using the following formula:

$$HED = Animal dose ( mg / k g ) multiplied by \frac{Animal Km}{Human Km}$$

The Km for a mouse is 3 and the Km for a human is 37.

Thus, for a human a 100 mg/kg dose in a mouse equates to a human dose of about 8.1 mg/kg.

### DETAILED DESCRIPTION

### Preparation of Botanically Derived Purified Cbd

The following describes the production of the botanically derived purified CBD which comprises greater than or equal to 98% w/w CBD and less than or equal to other cannabinoids was used in Example 1 below.

In summary the drug substance used is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CBD w/w, typically greater than 98% w/w.

The Cannabis sativa L. plants are grown, harvested, and processed to produce a botanical extract (intermediate) and then purified by crystallization to yield the CBD (botanically derived purified CBD).

The plant starting material is referred to as Botanical Raw Material (BRM); the botanical extract is the intermediate; and the active pharmaceutical ingredient (API) is CBD, the drug substance.

All parts of the process are controlled by specifications. The botanical raw material specification is described in Table A and the CBD API is described in Table B.

TABLE A
CBD botanical raw material specification

| Test | Method | Specification | |
|---|---|---|---|
| Identification: | | | |
| A | Visual | Complies | |
| B | TLC | Corresponds to standard (for | |
| | | CBD & CBDA) | |
| C | HPLC/UV | Positive for CBDA | |
| Assay: | In-house | NLT 90% of assayed | |
| CBDA + CBD | (HPLC/UV) | cannabinoids by peak area | |
| Loss on Drying | Ph. Eur. | NMT 15% | |
| Aflatoxin | UKAS method | NMT 4 | ppb |
| Microbial: | | | |
| TVC | Ph. Eur. | NMT10⁷ | cfu/g |
| Fungi | | NMT10⁵ | cfu/g |
| E. coli | | NMT10² | cfu/g |

| | | |
|---|---|---|
| Foreign Matter: | Ph. Eur. | NMT 2% |
| Residual Herbicides | Ph. Eur. | Complies |
| and Pesticides | | |

TABLE B
Specification of an exemplary botanically
derived purified CBD preparation

| | Test | | |
|---|---|---|---|
| Test | Method | Limits | |
| Appearance | Visual | Off-white/pale yellow crystals | |
| Identification | HPLC-UV | Retention time of major peak | |
| A | | corresponds to certified CBD | |
| | | Reference Standard | |
| Identification | GC-FID/MS | Retention time and mass spectrum | |
| B | | of major peak corresponds to | |
| | | certified CBD Reference Standard | |
| Identification | FT-IR | Conforms to reference spectrum | |
| C | | for certified CBD Reference Standard | |
| Identification | Melting | 65-67° | C. |
| D | Point | | |
| Identification | Specific | Conforms with certified CBD | |
| E | Optical | Reference Standard; −110° to −140° | |
| | Rotation | (in 95% ethanol) | |
| Total Purity | Calculation | ≥98.0% | |
| Chromatographic | HPLC-UV | ≥98.0% | |
| Purity 1 | | | |
| Chromatographic | GC-FID/MS | ≥98.0% | |
| Purity 2 | | | |
| CBDA | HPLC-UV | NMT 0.15% w/w | |
| CBDV | | NMT 1.0% w/w | |
| THC | | NMT 0.1% w/w | |
| CBD-C4 | | NMT 0.5% w/w | |
| Residual Solvents: | | | |
| Alkane | GC | NMT 0.5% w/w | |
| Ethanol | | NMT 0.5% w/w | |
| Residual Water | Karl | NMT 1.0% | w/w |
| | Fischer | | |

The purity of the botanically derived purified CBD preparation was greater than or equal to 98%. The botanically derived purified CBD includes THC and other cannabinoids, e.g., CBDA, CBDV, CBD-C1, and CBD-C4.

Distinct chemotypes of the Cannabis sativa L. plant have been produced to maximize the output of the specific chemical constituents, the cannabinoids. Certain chemovars produce predominantly CBD. Only the (−)-trans isomer of CBD is believed to occur naturally. During purification, the stereochemistry of CBD is not affected.

### Production of CBD Botanical Drug Substance

An overview of the steps to produce a botanical extract, the intermediate, are as follows:
a. Growing
b. Direct drying
c. Decarboxylation
d. Extraction—using liquid CO2
e. Winterization using ethanol
f. Filtration
g. Evaporation

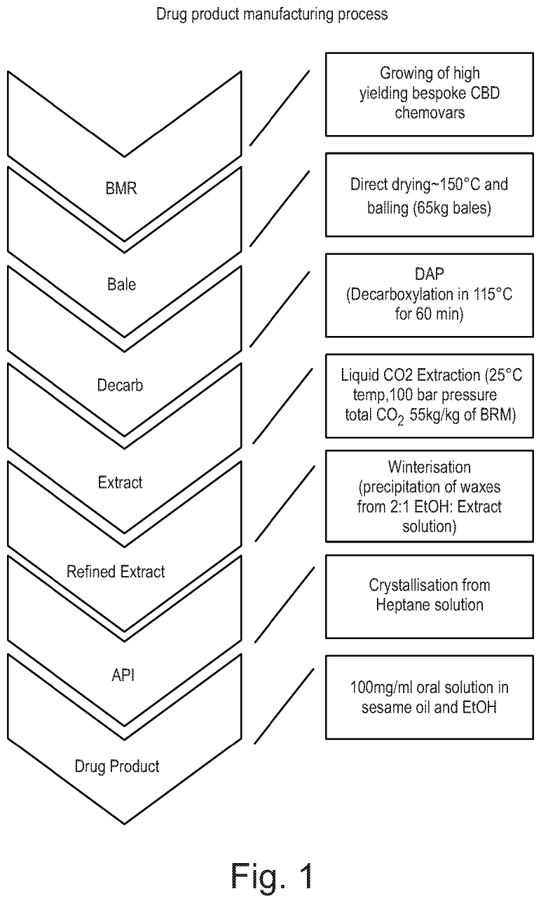

The process is described in greater detail below and in FIG. 1. High CBD yielding chemovars were grown, harvested, dried, baled and stored in a dry room until required. The botanical raw material (BRM) was finely chopped using an Apex mill fitted with a 1 mm screen. The milled BRM was stored in a freezer prior to extraction.

Decarboxylation of CBDA to CBD was carried out using heat. BRM was decarboxylated at 115° C. for 60 minutes.

Extraction was performed using liquid CO₂ at a temperature of 25° C. and 100 bar to produce botanical drug substance. The crude CBD BDS was winterized to refine the extract under standard conditions (2 volumes of ethanol at −20° C. for approximately 50 hours). The precipitated waxes were removed by filtration and the solvent was removed to yield the BDS.

### Production of Botanically Derived Purified CBD Preparation

The manufacturing steps to produce the botanically derived purified CBD preparation from BDS were as follows:
a. Crystallization using C5-C12 straight chain or branched alkane
b. Filtration
c. Vacuum drying

The BDS produced using the methodology above was dispersed in C5-C12 straight chain or branched alkane. The mixture was manually agitated to break up any lumps and the sealed container then placed in a freezer for approximately 48 hours. The crystals were isolated via vacuum filtration, washed with aliquots of cold C5-C12 straight chain or branched alkane, in this case heptane, and dried under a vacuum of <10 mb at a temperature of 60° C. until dry. The botanically derived purified CBD preparation was stored in a freezer at −20° C. in a pharmaceutical grade stainless steel container, with FDA food grade approved silicone seal and clamps.

### Physicochemical Properties of the Botanically Derived Purified CBD

The botanically derived purified CBD used in the clinical trial described in the invention comprises greater than or equal to 98% (w/w) CBD and less than or equal to 2% (w/w) of other cannabinoids. The other cannabinoids present are THC at a concentration of less than or equal to 0.1% (w/w); CBD-C1 at a concentration of less than or equal to 0.15% (w/w); CBDV at a concentration of less than or equal to 0.8% (w/w); and CBD-C4 at a concentration of less than or equal to 0.4% (w/w).

The botanically derived purified CBD used additionally comprises a mixture of both trans-THC and cis-THC. It was found that the ratio of the trans-THC to cis-THC is altered and can be controlled by the processing and purification process, ranging from 3.3:1 (trans-THC:cis-THC) in its unrefined decarboxylated state to 0.8:1 (trans-THC:cis-THC) when highly purified.

Furthermore, the cis-THC found in botanically derived purified CBD is present as a mixture of both the (+)-cis-THC and the (−)-cis-THC isoforms.

Clearly a CBD preparation could be produced synthetically by producing a composition with duplicate components.

Example 1 below describes the use of a botanically derived purified CBD in a rat model of temporal lobe epilepsy.

### EXAMPLE 1

### Cannabidiol (CBD) in a Rat Model of Temporal Lobe Epilepsy

This Example demonstrates the effect of botanically derived purified CBD and synthetic CBD in a rat model of temporal lobe epilepsy, Reduced Intensity Status Epilepticus (RISE).

### Methods

Reduced Intensity Status Epilepticus (RISE) model

The method, which detects anti-epileptic activity follows that described by Modebaze et al., 2016 (PLoS one, 11(2):e0147265).

On Day 1, the rats were treated with lithium chloride (127 mg/kg, 5 mL/kg, s.c.). Approximately 24 hours after, on Day 2, a-methyl scopolamine (1 mg/kg, 1 mL/kg, s.c.) was administered to reduce peripheral manifestations of muscarinic cholinergic receptor activation. Thirty minutes later, a dose of pilocarpine chloride was injected (range from 25 mg/kg×1 to 40 mg/kg×5, i.e. 25-200 mg/kg, 1 mL/kg, s.c., reference P6503, Sigma).

The rats were then observed for detecting signs of seizure and seizure severity was ranked using Racine's scale. The rats failing to reach Racine Stage 5 after the first injection were administered again with pilocarpine, up to a maximum of 3 injections at 7-30 minute intervals.

Status epilepticus was determined when seizure severity was equal to a score of 5 on a Racine's scale. Once this score was reached, the rats immediately received xylazine (2.5 mg/kg, intramuscularly, 1 mL/kg, Rompun at 2%).

Around one hour later, the rats received a “stop” solution containing MK-801 (0.1 mg/kg), Diazepam (2.5 mg/kg) and MPEP (20 mg/kg) subcutaneously at 1 mL/kg.

Behavioural signs of status epilepticus were ceased within 30 minutes and animals were then closely monitored during subsequent recovery. They were visually monitored until ambulatory and able to consume water and moistened, powdered food. During the recovery period, rats were kept on a heat pad to maintain body temperature. 5% glucose in physiological saline was given intraperitoneally to rehydrate the rats.

In most cases, recovery was nearly complete at 4 hours and all rats were fully recovered within 12 hours.

Hypromellose eye drops (Lacrigel) were used during status epilepticus to prevent subsequent ocular keratitis.

The rats were weighed at 24 and 48 hours then the rats were monitored until the beginning of the post-seizure behavioural battery. During the two weeks of recovery, some rats received 5% glucose in physiological saline.

### Post-Seizure Behavioural Battery (PSBB)

The lithium-pilocarpine rats were assessed in the two following behavioural tests twice weekly over the next 10 weeks, starting two weeks after status epilepticus induction (on Day 15). The PSBB and scoring system used here were based on that defined in Modebadze et al., 2016.

Pickup Task: The animal was picked up by grasping around the body. Responses were scored as follows:
1=Very easy pickup.
2=Easy pickup with vocalisation.
3=Some difficulties in pickup (rat reared and faced the hand).
4=Rat froze.
5=Difficulty pickup (rat moved away).
6=very difficult pickup (rat behaved defensively or attacked the hand).

Touch Task: The animal was gently prodded in the rump with a blunt instrument (e.g. scalpel handle). Responses were scored as follows:
1=No reaction.
2=Rat turned toward instrument.
3=Rat moved away from instrument.
4=Rat froze.
5=Rat turned toward the touch.
6=Rat turned away from the touch.
7=Rat jumped (with or without vocalisation).

For Cohort 2, the Li-Pilo rats were compared to the control rats for each task. No control rats were tested for Cohort 3.

The score obtained in these two tasks was multiplied to obtain the total score (for each PSBB test, the score of Touch Task (TT) was multiplied by the score of Pickup Task (PT)). Li-Pilo rats showing a total PSBB score 0 on four consecutive post-seizure behavioural battery (PSBB) tests, i.e., over a minimum of a 2 week period, were considered as rats developing spontaneous recurrent seizures.

From this point, the rats selected received vehicle during the first week of dosing then the test substance, CBD (SYN) or CBD (BOT 0.08%), at 200 mg/kg in drinking water until the end of the study, i.e. 7 weeks (1 week vehicle+6 weeks test substance). Dosing continued throughout the behavioural testing period (1 week). The drink was changed twice a week, until the end of the experiment.

The rats were weighed twice a week, at the same time that the vehicle/formulations were changed in the bottles. The glass bottles were also weighed twice a week for evaluating the volume drunk by the rat and adjusting the concentration during the experiment.

Water intake was also measured for control groups with measurement of body weight twice a week.

### Behavioural Tests

The behavioural tests started 6 weeks after the beginning of dosing, i.e. between weeks 11 and 13. The rats were tested in sub-groups, depending on the beginning of the treatment. The behavioural tests included the elevated plus maze, social investigation, social recognition and accelerating rotarod tests (tests done in this order on the week of assessment).

A PSBB test was done after the last behavioural test. Faeces, blood and brain were collected at the end of the experiment.

### Elevated Plus-Maze Test in the Rat

The method, which detects anxiolytic activity, follows that described by Handley and Mithani (Naunyn. Schmied. Arch. Pharmacol., 327, 1-5, 1984). Rodents avoid open spaces (the open arms of an elevated plus-maze). Anxiolytics increase exploratory activity in the open arms, as indicated by increased time spent on the open arms and/or by increased % open-arm entries.

The maze consisted of 4 arms of equal length and width (50×10 cm) arranged in the form of a plus sign (+). Two opposite arms were enclosed by 40 cm high walls (closed arms). The 2 other arms had no walls (open arms). The maze was raised approximately 65 cm above the floor. A rat was placed in the center of the plus-maze and was left to explore for 5 minutes. The number of entries into the open and closed arms and the time spent on the open arms were recorded. The % of open arm entries (open arm entries/total arm entries×100) was calculated.

A test of normality (D'Agostino and Pearson) was done to check the distribution of the data before applying the appropriate statistical analysis. Data on the total number of entries and on the percent of entries in open arms was analyzed using One-Way analysis of variance (ANOVA) followed by Sidak's multiple comparisons test. Data on the time spent in open arms was analysed using Kruskal-Wallis Test followed by Dunn's multiple comparisons test.

### Social Investigation Test in the Rat

Deficits in social investigation induced by antagonists of glutamate receptor of the NMDA type such as dizocilpine (MK-801) are considered as an animal model of negative symptoms of schizophrenia, as described by Sams-Dodd (Rev. Neurosci, 10, 59-90, 1999). The method follows that described by Koros et al (Neuropsychopharmacology, 32, 562-576, 2007).

Two unfamiliar rats (housed in 2 separate cages) from the same group were placed in an unfamiliar testing arena (80×80×40 cm) under dim light (<100 Lux). The behavior of the 2 rats was recorded for 10 minutes.

The time each rat spent investigating (sniffing, grooming, licking, closely following) the other rat was measured manually for 10 minutes. Locomotor activity (line crossing) was also recorded.

A test of normality (D'Agostino and Pearson) was done to check the distribution of the data before applying the appropriate statistical analysis. Data on the investigation duration was analyzed using Kruskal-Wallis Test followed by Dunn's multiple comparisons test. Data on the number of crossings was analysed using One-Way ANOVA followed by Sidak's multiple comparisons test.

### Accelerating Rotarod Test in the Rat

The method, which detects neurological deficits, follows that described by Dunham and Miya (J. Am. Pharm. Ass. 46, 208-209, 1957) and adapted by Jones and Roberts (J. Pharm. Pharmacol. 20, 302-304, 1968).

Training sessions: the rats were placed on a rod (diameter: 7 cm) rotating at a constant speed of 4 revolutions per minute for a 2-minute period. If they fell off during this period they were replaced on the rod. This training procedure was repeated 2 times.

Test sessions: starting at least 2 hours after the training sessions, rats were placed on the rotarod for a maximum period of 3 minutes. The rod rotated at a constant speed of 4 revolutions per minute at the beginning of the test and it was progressively accelerated up to 30 revolutions per minute at the end of the test. The number of animals which fell off before the end of this period was counted and the latency to fall off was recorded (cut-off: 3 minutes). The testing procedure was replicated 2 times for each animal.

The data were analyzed separately for each test session. The mean performance over the 2 test sessions was also calculated and analyzed.

A test of normality (D'Agostino and Pearson) was done to check the distribution of the data before applying the appropriate statistical analysis. Data was analyzed using Kruskal-Wallis Test followed by Dunn's multiple comparisons test for each individual session; the mean of both test sessions was analysed using One-Way ANOVA followed by Sidak's multiple comparisons test.

### PSBB

The PSBB score was analysed pre and post-treatment to assess firstly if the animals were evenly distributed in the 3 different treatment groups based on their epileptic phenotype and if it was affected differently by synthetic and botanical CBD. The D'Agostino and Pearson normality test was used and the data analysed with a One-Way ANOVA followed by Sidak's multiple comparisons test.

The average daily dose received by the two treated groups was compared with a student t-test as the data was normally distributed as shown by the D'Agostino and Pearson test.

The percentage change of the PSBB score between the start (average of PSBB scores measured on week 1 and 2) and the end of the treatment period (measured on week 11, 12 or 13) was compared between the groups treated with synthetic and botanical CBD using the Man-Whitney test as it was non normally distributed as shown by the D'Agostino and Pearson test.

### Results

Reduced Intensity Status Epilepticus model in the Rat
Induction Phase with Administration of Lithium-Pilocarpine (Li-Pilo)

A total of 150 rats was included in the whole experiment. Lithium followed by pilocarpine were administered in 140 rats, 90 rats during Phase 1 on 4 consecutive days with 18 to 25 rats tested per day and 50 rats during Phase 2 with 25 rats per day.

Seizure of Racine stage 5 was observed in 1 rat after one administration of pilocarpine, in 44 rats after two administrations of pilocarpine and in 28 rats after 3 administrations of pilocarpine.

Seventy-three rats received the stop solution. Forty-eight rats were sacrificed as they did not reach the selection criteria.

During this phase, 19 rats died after administration of lithium-pilocarpine and/or the stop solution.

During the recovery period, i.e. between lithium-pilocarpine administration and the first post-seizure behavioural battery, 13 rats died (10 rats during Phase 1 and 3 rats during Phase 2).

### General Status/Body Weight

Phase 1:

Ten rats were included in the control group. The body weight of rats increased from week 5 to week 11, indicating normal growth (from 300 g on week 5 to 379 g on week 14). One rat did not gain a lot of weight and remained small during the 7 weeks (Rat N° 50, +32 g), indicating that liquid consumption was low due to the vehicle taste. The other 9 rats gained between 56 and 115 g in 7 weeks.

Nine rats were included in the control Li-pilocarpine (Li-Pilo) group. The body weight of rats increased from week 5 to week 13, indicating normal growth (from 260 g on Week 5 to 415 g on Week 13).

Nine rats were included in the Li-Pilo rats treated with synthetic CBD group. Two rats died (Rats N° 46 and 16). Three other rats lost weight on the 7 weeks of test (rats N° 43 (−74 g), 79 (−30 g) and 100 (−4 g)). The body weight of the other 4 rats slightly increased from +19 g to +52 g on Weeks 11/13 as compared with Week 5.

Ten rats were included in the Li-Pilo rats treated with botanical CBD group. Four rats lost weight on the 7 weeks of test (rats N° 48 (−1 g), 53 (−51 g), 26 (−46 g) and 39 (−12 g)). The body weight of the other 6 rats increased from +4 g to +59 g on Weeks 11/13 as compared with Week 5.

### Phase 2:

Two rats were included in the control Li-pilocarpine (Li-Pilo) group. The body weight of rats increased from week 5 to week 13, indicating normal growth (from 251 g on Week 5 to 426 g on Week 13).

Thirteen rats were included in the Li-Pilo rats treated with synthetic CBD group. One rat was sacrificed for ethical reasons before the end of the experiment (Rat N° 146). Four other rats lost weight on the 7 weeks of test (rats N° 138 (−19 g), N° 139 (−29 g), 150 (−3g) and 101 (−64g)). The body weight of the 8 other rats slightly increased from +1 g to +77 g on Weeks 11/13 as compared with Week 5.

Four rats were included in Li-Pilo rats treated with botanical CBD group. Two rats lost weight during the 7 weeks of test (rats N° 106 (−56 g), and N° 145 (−4 g). The body weight of the 2 other rats increased from +32 g to +79 g on Weeks 11/13 as compared with Week 5.

Mice were balanced across the different treatment groups by their body weight, hence no significant differences were observed (one-way ANOVA) in body weights among the groups prior to commencing the study.

Post hoc multiple comparisons indicated that treatment had no significant effect on the increase in body weight over time.

### Post-Seizure Behavioural Battery (PSBB)

### Pre-treatment

### Phase 1:

In control rats, the total PSBB score was between 1.0±0.0 and 1.6±0.5 from week 2 to

week 5.

In the Li-Pilo rats, the PSBB score was clearly increased from week 2 to week 5, as compared with control rats (values between 13.4 and 20.3). Seventeen rats were selected after week 3, six rats after week 4 and five rats after week 5; i.e. a total of 28 rats that showed a PSBB score 0 on two consecutive weeks, out of the 35 rats tested.

During this phase, one rat died (Rat N° 34).

### Phase 2:

In the Li-Pilo rats, the PSBB score was between 12.5±2.4 and 22.8±2.2 from week 2 to week 5. Fourteen rats were selected after week 3 and 5 rats after week 5; i.e. a total of 19 rats that showed a PSBB score on two consecutive weeks, out of the 28 rats tested.

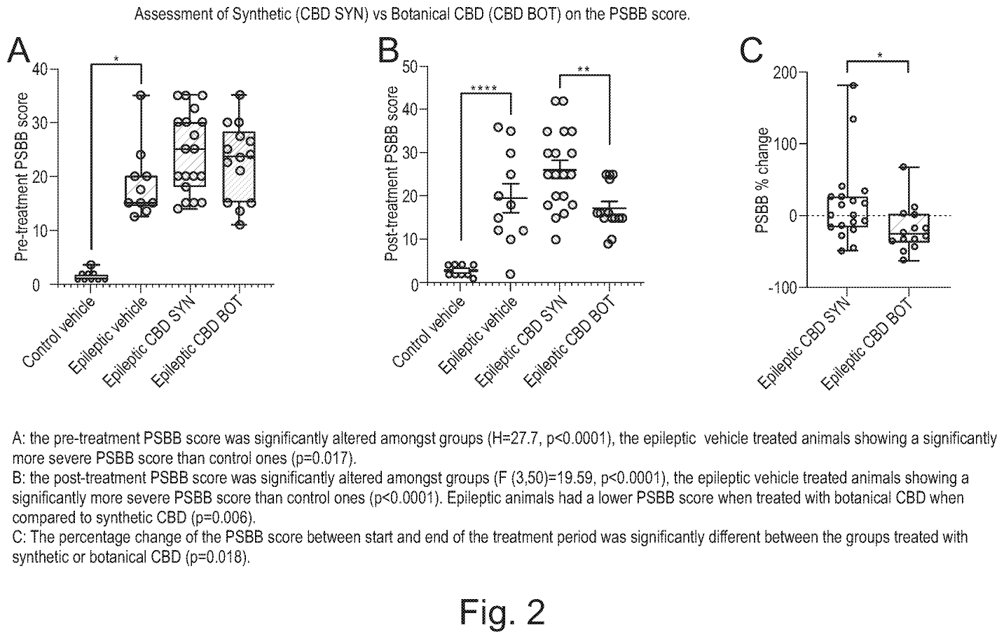

In control rats, the pre-treatment PSBB score for Phase 1 and Phase was 1.4±0.2 (FIG. 2A). In control Li-Pilo group, this was significantly increased compared with control rats (+16.9, p<0.05). The pre-treatment PSBB score was not significantly different between the epileptic groups of rats subsequently allocated to one of the 3 treatments.

### Post-Treatment:

In control rats, the post-treatment PSBB score was 2.8±0.3 (FIG. 2B).

In the control Li-Pilo group, the post-treatment PSBB score was significantly increased, as compared with control rats (+16.7, p<0.0001).

Synthetic CBD (200 mg/kg, given orally for 6/7 weeks before the test), did not significantly affect the post-treatment PSBB score, as compared with control Li-Pilo rats (epileptic vehicle).

Botanical CBD (200 mg/kg, given orally for 6/7 weeks before the test), did not significantly affect the pre-treatment PSBB score, as compared with control Li-Pilo rats.

The post-treatment PSBB score was significantly lower in the epileptic group treated with botanical CBD when compared to the animals treated with the synthetic compound (−8.9, p<0.01).

When the percentage of change of the PSBB score between the pre and post-treatment periods was analysed, it appeared that botanical CBD was significantly more potent in decreasing this score than the synthetic compound (p<0.05, FIG. 2C).

No correlation between the doses of CBD received and the percentage of change of the PSBB scores was observed (CBD SYN: Pearson r=0.033, p=0.89; CBD BOT: Pearson r=−0.086, p=0.76).

### Elevated Plus Maze Test

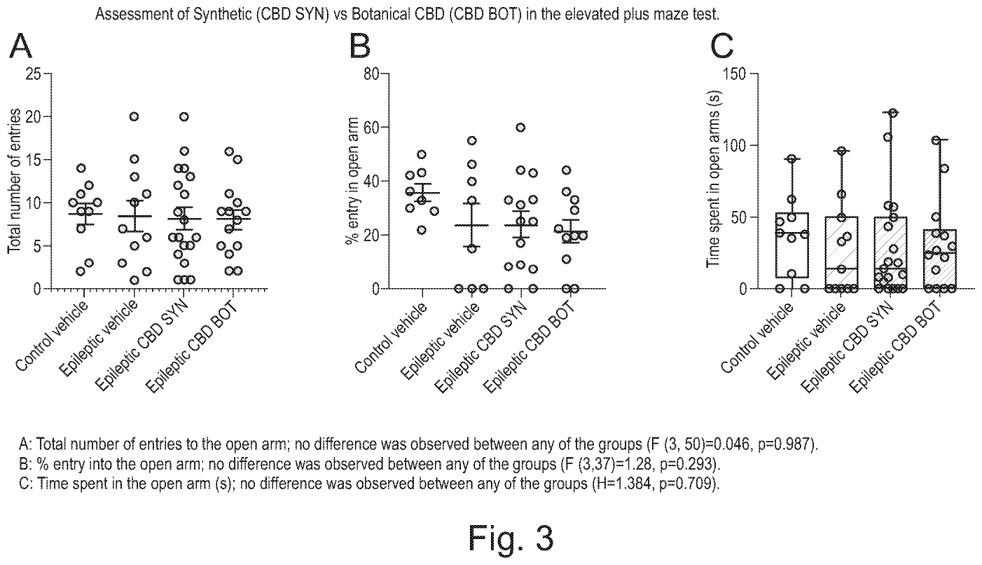

In the control group, the total number of entries in 5 minutes was 8.7±1.2 (FIG. 3A). The percent of entries in open arms was of 35.6±3.2% (FIG. 3B) and the time spent in open arms was of 37.4±9.0 seconds (FIG. 3C).

In control Li-Pilo (epileptic vehicle) group, the total number of entries was similar to the control group. The percent of entries and the time spent in open arms were decreased but the values were not statistically significant, as compared with control group.

Synthetic CBD (200 mg/kg, given orally for 6/7 weeks before the test), did not clearly affect the total number of entries, the percent of entries in open arms or the time spent in open arms, as compared with control Li-Pilo rats.

Botanical CBD (200 mg/kg, given orally for 6/7 weeks before the test), did not clearly affect the total number of entries, the percent of entries in open arms or the time spent in open arms, as compared with control Li-Pilo rats.

### Social Investigation Test in the Rat

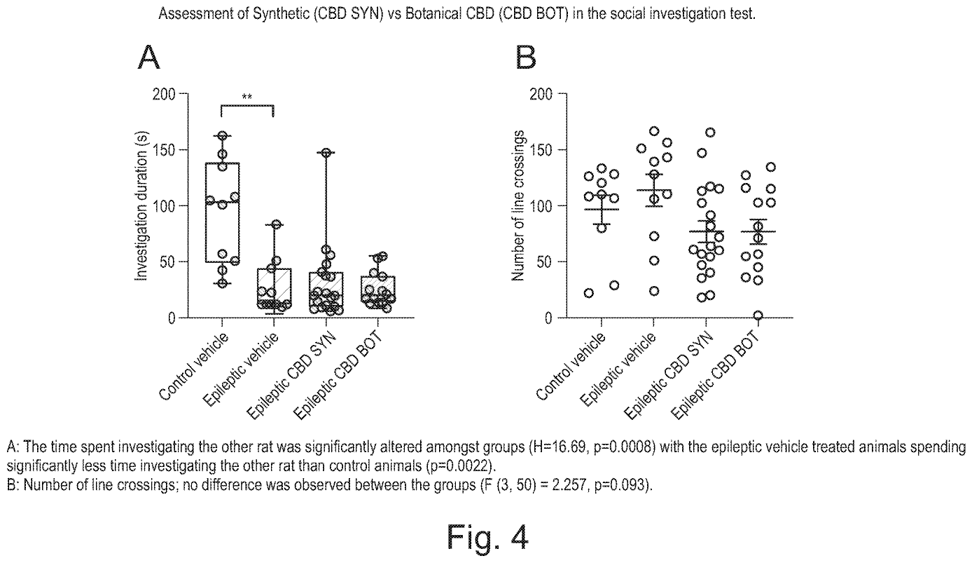

In the control group, the duration of investigation was 93.9±14.6 seconds (FIG. 4A) and the number of line crossings 96.3±12.7 (FIG. 4B).

In the control Li-Pilo (epileptic vehicle) group, the duration of investigation was clearly decreased, as compared with control rats (−71%, p<0.01). The number of line crossings was not significantly changed, as compared to control rats.

Synthetic CBD (200 mg/kg, given orally for 6/7 weeks before the test), did not significantly change the duration of investigation or the number of line crossings, as compared with control Li-Pilo rats.

Botanical CBD (200 mg/kg, given orally for 6/7 weeks before the test), did not significantly change the duration of investigation or the number of line crossings, as compared with control Li-Pilo rats.

### Conclusion

The botanically derived purified CBD significantly reduced the post-treatment PSBB score, whereas the synthetic CBD failed to lower the score in the epileptic animals. Hence there is a difference in the potency of the two materials, with botanically derived purified being more efficacious than synthetic CBD.

The PSBB scoring system has been validated as a useful measure to evaluate epileptic animals (Modebadze et al., 2016) and as such the ability of a compound to reduce the PSBB after treatment is suggestive of the ability of the compound to have efficacy in the treatment of temporal lobe epilepsy.

Such data are significant as they demonstrate that such a CBD composition may be useful in the treatment of epilepsy.

1. A method of treating temporal lobe seizures (TLE) comprising administering a cannabidiol (CBD) preparation to a subject in need thereof.

2. The method according to claim 1, wherein the CBD preparation comprises greater than or equal to 98% (w/w) CBD and less than or equal to 2% (w/w) other cannabinoids, wherein the less than or equal to 2% (w/w) other cannabinoids comprise the cannabinoids tetrahydrocannabinol (THC);
cannabidiol-C1 (CBD-C1); cannabidivarin (CBDV); and cannabidiol-C4 (CBD-C4), and wherein the THC is present as a mixture of trans-THC and cis-THC.

3. The method according to claim 1, wherein the CBD is present is isolated from cannabis plant material.

4. The method according to claim 2, wherein at least a portion of at least one of the cannabinoids present in the CBD preparation is isolated from cannabis plant material.

5. The method according to claim 1, wherein the CBD is present as a synthetic preparation.

6. The method according to claim 5, wherein at least a portion of at least one of the cannabinoids present in the CBD preparation is prepared synthetically.

7. The method according to claim 1, wherein the dose of CBD is greater than 5 mg/kg/day.

8. The method according to claim 1, wherein the dose of CBD is 20 mg/kg/day.

9. The method according to claim 1, wherein the dose of CBD is 25 mg/kg/day.

10. The method according to claim 1, wherein the dose of CBD is 50 mg/kg/day.

11. (canceled)
